(19) United States
(12) Patent Application Publication
(10) Pub. No.: US 2019/0013092 A1
(43) Pub. Date: Jan. 10, 2019

(54) SYSTEM AND METHOD FOR FACILITATING DETERMINATION OF A COURSE OF ACTION FOR AN INDIVIDUAL

(71) Applicant: KONINKLIJKE PHILIPS N.V., EINDHOVEN (NL)

(72) Inventors: Aart Tijmen VAN HALTEREN, Geldrop (NL); Tarsem SINGH, Cambridge (GB); Monica JIANU, Cambridge (GB); Nuwani Ayantha EDIRISINGHE, Newmarket (GB)

(21) Appl. No.: 16/019,041

(22) Filed: Jun. 26, 2018

Related U.S. Application Data
(60) Provisional application No. 62/528,608, filed on Jul. 5, 2017.

(51) Int. Cl. G16H 20/70; G16H 50/20; G09B 5/02; G09B 19/00; A61B 5/0205; A61B 5/16

(52) U.S. Cl. CPC G16H 20/70; G16H 50/20; G09B 5/02; A61B 5/024; A61B 5/0205; A61B 5/165; G09B 19/00

(57) ABSTRACT

The present disclosure pertains to a system for facilitating determination of a course of action for a subject. In some embodiments, the system obtains sensor-generated output signals conveying information related to interactions between the subject and a consultant during a consultation period; detects a mood of the subject; determines a course of action for the subject during the consultation period based on the detected mood; and provides, via a user interface, one or more cues for presentation to the consultant during the consultation period, the cues indicating the determined course of action to be taken by the consultant for interacting with the subject.

### CROSS-REFERENCE TO PRIOR APPLICATIONS

This application claims the benefit of U.S. Provisional Application No. 62/528,608, filed on 5 Jul. 2017. This application is hereby incorporated by reference herein.

### BACKGROUND

### 1. Field

The present disclosure pertains to a system and method for facilitating determination of a course of action for an individual.

### 2. Description of the Related Art

Health coaching is commonly used to help patients self-manage their chronic diseases and elicit behavior change. Coaching techniques used during coaching may include motivational interviewing and goal setting. Although computer-assisted coaching systems exist, such systems may not facilitate an objective assessment of a quality of an individual coaching session. For example, prior art systems may present educational information and set one or more care plan goals without accounting for the patients' psychosocial needs. These and other drawbacks exist.

### SUMMARY

Accordingly, one or more aspects of the present disclosure relate to a system configured to facilitate determination of a course of action for a subject. The system comprises one or more sensors configured to generate, during a consultation period, output signals conveying information related to interactions between the subject and a consultant; one or more processors; or other components. The one or more sensors include at least a sound sensor and an image sensor. The one or more processors are configured by machine-readable instructions to: obtain, from the one or more sensors, the sensor-generated output signals during the consultation period; detect, based on the sensor-generated output signals, a mood of the subject during the consultation period; determine a course of action for the subject during the consultation period based on the detected mood; and provide, via a user interface, one or more cues for presentation to the consultant during the consultation period, the cues indicating the determined course of action to be taken by the consultant for interacting with the subject.

Yet another aspect of the present disclosure relates to a method for facilitating determination of a course of action for a subject with a system. The system comprises one or more sensors, one or more processors, or other components. The method comprises: obtaining, from the one or more sensors, output signals conveying information related to interactions between the subject and a consultant during a consultation period, the one or more sensors including at least a sound sensor and an imaging sensor; detecting, based on the sensor-generated output signals, a mood of the subject during the consultation period; determining, with the one or more processors, a course of action for the subject during the consultation period based on the detected mood; and providing, via a user interface, one or more cues for presentation to the consultant during the consultation period, the cues indicating the determined course of action to be taken by the consultant for interacting with the subject.

Still another aspect of present disclosure relates to a system for facilitating determination of a course of action for an individual. The system comprises: means for generating, during a consultation period, output signals conveying information related to interactions between the subject and a consultant, the means for generating including at least a sound sensor and an imaging sensor; means for obtaining the output signals during the consultation period; means for detecting, based on the output signals, a mood of the subject during the consultation period; means for determining a course of action for the subject during the consultation period based on the detected mood; and means for providing one or more cues for presentation to the consultant during the consultation period, the cues indicating the determined course of action to be taken by the consultant for interacting with the subject.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system for facilitating determination of a course of action for a subject, in accordance with one or more embodiments.

FIG. 2 illustrates a patient coaching summary, in accordance with one or more embodiments.

FIG. 3 illustrates a method for facilitating determination of a course of action for a subject, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

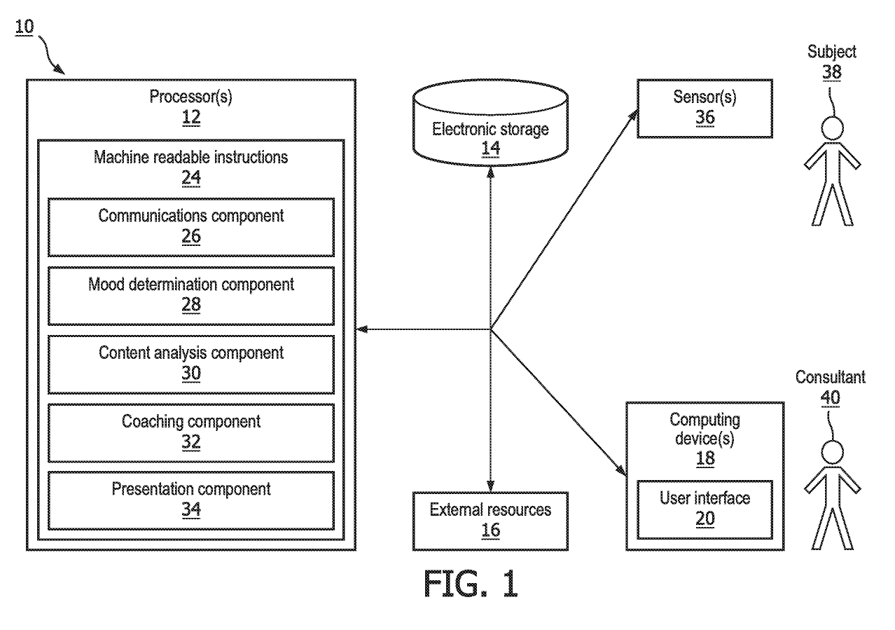

FIG. 1 is a schematic illustration of a system 10 for facilitating determination of a course of action for an individual. In some embodiments, system 10 facilitates means for supporting one or more health coaches (or other individuals) before, during, and after a visit with a patient (or other individual). In some embodiments, a health coach may encounter one or more problems before meeting with a patient. For example, these problems may include (i) a large amount of time spent travelling from one patient to another, leaving very little time to prepare for the session, (ii) lack of a means to quickly digest notes obtained during a session on the go, (iii) a need for one health coach to know what was discussed in the previous consultation by other health coaches, (iv) primary means of learning includes experience in the field, and (v) due to staff shortages, health coaches need to start working without receiving adequate training. In some embodiments, the health coaches may encounter one or more problems while meeting with the patient. For example, these problems may include (i) an inexperienced coach misinterpreting the mood of the conversation, thus failing to establish a rapport with the patient, (ii) the health coach not feeling confident about the actions the patient can take to achieve a certain health goal, and (iii) the health coaches failing to provide the right type of information, which may affect the confidence the patient has in the health coaches. In some embodiments, the health coaches may encounter one or more problems after meeting with the patient. For example, these problems may include lack of a means to objectively assess the quality of an individual coaching session.

In some embodiments, system 10 facilitates provision of a brief audio summary of one or more previous interactions with a subject to a consultant. For example, the audio summary may be provided prior to a coach visiting a patient (e.g., during the drive and/or waiting for the patient to arrive). In some embodiments, system 10 detects, via voice recognition, one or more keywords and/or phrases discussed during one or more interactions with the subject. In some embodiments, system 10 is configured to perform, based on the one or more keywords and/or phrases) a semantic search in a coaching database. In some embodiments, system 10 is configured to deliver suggestions that are relevant for the topic of an interaction session on a screen which the consultant may then follow. In some embodiments, the consultant's field of view is augmented with the relevant suggestions. In some embodiments, system 10 is configured to determine a mood of the subject and suggest alternative tactics in the goal setting dialogues responsive to the subject not responding well to an approach taken.

In some embodiments, system 10 comprises one or more processors 12, electronic storage 14, external resources 16, computing device 18, one or more sensors 36, or other components.

In some embodiments, one or more sensors 36 are configured to generate, during a consultation period, output signals conveying information related to interactions between subject 38 and consultant 40. In some embodiments, one or more sensors 36 include at least a sound sensor and an image sensor. In some embodiments, the sound sensor includes a microphone and/or other sound sensing/recording devices configured to generate output signals related to one or more verbal features (e.g., tone of voice, volume of voice, etc.) corresponding to subject 38. In some embodiments, the image sensor includes one or more of a video camera, a still camera, and/or other cameras configured to generate output signals related to one or more facial features (e.g., eye movements, mouth movements, etc.) corresponding to subject 38. In some embodiments, one or more sensors 36 include a heart rate sensor, a respiration sensor, a perspiration sensor, an electrodermal activity sensor, an activity sensor (e.g., seat activity sensor), and/or other sensors.

In some embodiments, one or more sensors 36 are implemented as one or more wearable devices (e.g., wrist watch, patch, Apple Watch, Fitbit, Philips Health Watch, etc.). In some embodiments, information from one or more sensors 36 may be automatically transmitted to computing device 18, one or more remote servers, or other destinations via one or more networks (e.g., local area networks, wide area networks, the Internet, etc.) on a periodic basis, in accordance to a schedule, or in response to other triggers.

Electronic storage 14 comprises electronic storage media that electronically stores information (e.g., a patient profile indicative of psychosocial needs of subject 38.). The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, processor 12, etc.). In some embodiments, electronic storage 14 may be located in a server together with processor 12, in a server that is part of external resources 16, in a computing device 18, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processor 12, information received via computing devices 18 and/or graphical user interface 20 and/or other external computing systems, information received from external resources 16, and/or other information that enables system 10 to function as described herein.

External resources 16 include sources of information and/or other resources. For example, external resources 16 may include subject 38's electronic coaching record (ECR), subject 38's electronic health record (EHR), or other information. In some embodiments, external resources 16 include health information related to subject 38. In some embodiments, the health information comprises demographic information, vital signs information, medical condition information indicating medical conditions experienced by subject 38, treatment information indicating treatments received by subject 38, and/or other health information. In some embodiments, external resources 16 include sources of information such as databases, websites, etc., external entities participating with system 10 (e.g., a medical records system of a health care provider that stores medical history information of patients), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. External resources 16 may be configured to communicate with processor 12, computing device 18, electronic storage 14, and/or other components of system 10 via wired and/or wireless connections, via a network (e.g., a local area network and/or the internet), via cellular technology, via Wi-Fi technology, and/or via other resources. In some embodiments, some or all of the functionality attributed herein to external resources 16 may be provided by resources included in system 10.

Computing devices 18 are configured to provide an interface between consultant 40 and/or other users, and system 10. In some embodiments, individual computing devices 18 are and/or are included in desktop computers, laptop computers, tablet computers, smartphones, smart wearable devices including augmented reality devices (e.g., Google Glass) and wrist-worn devices (e.g., Apple Watch), and/or other computing devices associated with consultant 40, and/or other users. In some embodiments, individual computing devices 18 are, and/or are included in equipment used in hospitals, doctor's offices, and/or other facilities. Computing devices 18 are configured to provide information to and/or receive information from subject 38, consultant 40, and/or other users. For example, computing devices 18 are configured to present a graphical user interface 20 to subject 38, consultant 40, and/or other users to facilitate entry and/or selection of information related to psychosocial needs of subject 38. In some embodiments, graphical user interface 20 includes a plurality of separate interfaces associated with computing devices 18, processor 12, and/or other components of system 10; multiple views and/or fields configured to convey information to and/or receive information from subject 38, consultant 40, and/or other users; and/or other interfaces.

In some embodiments, computing devices 18 are configured to provide user interface 20, processing capabilities, databases, or electronic storage to system 10. As such, computing devices 18 may include processor 12, electronic storage 14, external resources 16, or other components of system 10. In some embodiments, computing devices 18 are connected to a network (e.g., the internet). In some embodiments, computing devices 18 do not include processor 12, electronic storage 14, external resources 16, or other components of system 10, but instead communicate with these components via the network. The connection to the network may be wireless or wired. For example, processor 12 may be located in a remote server and may wirelessly cause presentation of the determined course of action via the user interface to a care provider on computing devices 18 associated with that caregiver (e.g., a doctor, a nurse, a health coach, etc.).

Examples of interface devices suitable for inclusion in user interface 20 include a camera, a touch screen, a keypad, touch sensitive or physical buttons, switches, a keyboard, knobs, levers, a display, speakers, a microphone, an indicator light, an audible alarm, a printer, tactile haptic feedback device, or other interface devices. The present disclosure also contemplates that computing devices 18 includes a removable storage interface. In this example, information may be loaded into computing devices 18 from removable storage (e.g., a smart card, a flash drive, a removable disk, etc.) that enables caregivers or other users to customize the implementation of computing device 18. Other exemplary input devices and techniques adapted for use with Computing devices 18 or the user interface include an RS-232 port, RF link, an IR link, a modem (telephone, cable, etc.), or other devices or techniques.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device 18, devices that are part of external resources 16, electronic storage 14, or other devices.)

In some embodiments, processor 12, external resources 16, computing devices 18, electronic storage 14, one or more first sensors 34, one or more second sensors 36, and/or other components may be operatively linked via one or more electronic communication links. For example, such electronic communication links may be established, at least in part, via a network such as the Internet, and/or other networks. It will be appreciated that this is not intended to be limiting, and that the scope of this disclosure includes embodiments in which these components may be operatively linked via some other communication media. In some embodiments, processor 12 is configured to communicate with external resources 16, computing devices 18, electronic storage 14, and/or other components according to a client/server architecture, a peer-to-peer architecture, and/or other architectures.

As shown in FIG. 1, processor 12 is configured via machine-readable instructions 24 to execute one or more computer program components. The computer program components may comprise one or more of a communications component 26, a mood determination component 28, a content analysis component 30, a coaching component 32, a presentation component 34, or other components. Processor 12 may be configured to execute components 26, 28, 30, 32, or 34 by software; hardware; firmware; some combination of software, hardware, or firmware; or other mechanisms for configuring processing capabilities on processor 12.

It should be appreciated that although components 26, 28, 30, 32, and 34 are illustrated in FIG. 1 as being co-located within a single processing unit, in embodiments in which processor 12 comprises multiple processing units, one or more of components 26, 28, 30, 32, or 34 may be located remotely from the other components. The description of the functionality provided by the different components 26, 28, 30, 32, or 34 described below is for illustrative purposes, and is not intended to be limiting, as any of components 26, 28, 30, 32, or 34 may provide more or less functionality than is described. For example, one or more of components 26, 28, 30, 32, or 34 may be eliminated, and some or all of its functionality may be provided by other components 26, 28, 30, 32, or 34. As another example, processor 12 may be configured to execute one or more additional components that may perform some or all of the functionality attributed below to one of components 26, 28, 30, 32, or 34.

Communications component 26 is configured to obtain, from one or more sensors 36, the sensor-generated output signals during the consultation period. In some embodiments, communications component 26 is configured to continuously obtain the sensor-generated output signals (e.g., on a periodic basis, in accordance with a schedule, or based on other automated triggers). In some embodiments, subject 38 includes one or more of a patient, an employee, a customer, a client, and/or other subjects. In some embodiments, consultant 40 includes one or more of a health care professional (e.g., a doctor, a nurse, a health coach), a manager, a sales consultant, an attorney, a realtor, a financial advisor, and/or other consultants.

In some embodiments, communications component 26 is configured to obtain one or more of demographics information associated with subject 38, clinical information associated with subject 38, psychosocial needs associated with subject 38, information related to subject 38's phenotype, disease impact associated with subject 38, subject 38's comfort with technology, coping style associated with subject 38, social support information associated with subject 38, self-care abilities of subject 38, patient activation information associated with subject 38, and/or other information. In some embodiments, communications component 26 is configured to obtain the information associated with subject 38 via a survey, a query, data provided by external resources 16 (e.g., electronic health records), data stored on electronic storage 14, and/or via other methods.

In some embodiments, communications component 26 is configured to receive, from one or more sensors 36, a live view of a real-world environment. In some embodiments, the received live view may be a still image or part of a sequence of images, such as a sequence in a video stream.

Mood determination component 28 is configured to detect, based on the sensor-generated output signals, a mood of subject 38. The mood may indicate an emotion or feeling of subject 38. For example, the mood of subject 38 may include one or more levels of happiness, sadness, seriousness, anger, energeticness, irritability, stress, fatigue, and/or other states. The mood may be invoked based on an event (e.g., an event that occurs during the interaction with consultant 40). In some embodiments, mood determination component 28 is configured to detect the mood of subject 38 based on one or more of a tone of voice of subject 38, verbal cues, facial expressions of subject 38, seat activities of subject 38, a heart rate of subject 38, a respiration of subject 38, a perspiration of subject 38, an electrodermal activity of subject 38, and/or other information.

In some embodiments, mood determination component 28 is configured to determine the mood of subject 38 based on one or more of a volume, an intonation, a speed and/or other features of subject 38's speech. In some embodiments, subject 38's speech features include one or more of stuttering, dry throat/loss of voice, shaky voice, and/or other features. In some embodiments, mood determination component 28 is configured to compare one or more verbal features corresponding to subject 38 with a voice database (e.g., a database comprising speech rate, voice pitch, voice tone and/or other verbal features associated with emotions, moods, and/or other psychological characteristics) to determine the mood of subject 38. For example, responsive to subject 38's speaking volume being decreased, mood determination component 28 may determine that subject 38 is feeling overwhelmed.

In some embodiments, mood determination component 28 is configured to analyze facial expressions of subject 38 by extracting features of subject 38's face. In some embodiments, mood determination component 28 is configured to compare the extracted features with a facial recognition database (e.g., a database comprising facial features and expressions associated with emotions, moods, and/or other psychological characteristics) to determine the mood of subject 38. In some embodiments, different features including one or more of regions around the eyes, the mouth, and/or other regions may be extracted. For example, responsive to a detection of rapid eye twitches along with a raised voice, mood determination component 28 may determine that subject 38 is agitated.

As another example, mood determination component 28 may (i) predefine one or more word categories (e.g., emotion words) in a database, (ii) determine a proportion of words in a coaching session transcript of subject 38 that correspond to the one or more word categories, and (iii) determine the mood of subject 38 based on the determined proportion. In this example, subject may be using the word “sad” and/or other words synonymous with “sad” approximately 40 percent of the time during the coaching session. As such, mood determination component 28 may determine that subject 38's overall mood with respect to a particular treatment and/or lifestyle is sad (e.g., negative). In some embodiments, responsive to subject 38's repeated use of words associated with emotions (e.g., depressed, suicidal, lonely, helpless, etc.) or words associated with symptoms (e.g., breathless, cough, fever, side effects, etc.), mood determination component 28 is configured to determine that subject 38's overall mood with respect to a particular treatment and/or lifestyle is despondent.

In some embodiments, mood determination component may be and/or include a prediction model. As an example, the prediction model may include a neural network or other prediction model (e.g., machine-learning-based prediction model or other prediction model) that is trained and utilized for determining the mood of subject 38 and/or other parameters (described above). As an example, if a neural network is used, the neural network may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion. By way of a non-limiting example, mood determination component 28 may determine the mood of subject 38 based on a specific physiological or behavioral characteristic possessed by subject 38. In this example, mood determination component 28 may associate a particular mood with a pattern of specific physiological or behavioral characteristics associated with subject 38.

Content analysis component 30 is configured to perform semantic analysis on the sensor-generated output signals to detect one or more words or phrases expressed during the interactions between subject 38 and consultant 40. In some embodiments, content analysis component 30 is configured to detect one or more keywords discussed during the interactions with subject 38. In some embodiments, the sensor-generated output signals include audio signals (e.g., sounds). In some embodiments, content analysis component 30 is configured to isolate segments of sound that likely to be speech and convert the segments into a series of numeric values that characterize the vocal sounds in the output signals. In some embodiments, content analysis component 30 is configured to match the converted segments to one or more speech models. In some embodiments, the one or more speech models include one or more of an acoustic model, a lexicon, a language model, and/or other models. In some embodiments, the acoustic model represents acoustic sounds of a language and may facilitate recognition of the characteristics of subject 38, consultant 40, and/or other individuals' speech patterns and acoustic environments. In some embodiments, the lexicon includes a database of words in a language along with information related to the pronunciation of each word. In some embodiments, the language model facilitates determining ways in which the words of a language are combined. In some embodiments, content analysis component 30 matches an audio pattern to a preloaded phrase and/or keyword. In some embodiments, content analysis component 30 facilitates determination of one or more words or phrases based an audio foot print of individual components of each word (e.g., utterance, vowels, etc.).

In some embodiments, responsive to a detection of one or more words or phrases, content analysis component 30 is configured to perform a semantic search in one or more databases provided by electronic storage 14, external resources 16, and/or other databases. As an example, the database may include a coaching database. In some embodiments, content analysis component 30 performs the semantic search to facilitate determining one or more suggestions for a course of action to be taken by consultant 40 for interacting with subject 38. For example, the one or more suggestions may include one or more topics for a coaching session.

Coaching component 32 is configured to determine a course of action for the consultation period for interacting with subject 38. In some embodiments, the course of action is determined during the consultation period based on the detected mood, the one or more words or phrases, and/or other information. In some embodiments, coaching component 32 is configured to determine the course of action at any time (e.g., continuously, in the beginning, every 15 minutes, responsive to a change in the detected mood, and/or any other period) during the consultation period. In some embodiments, coaching component 32 is configured to determine the course of action one or more times (e.g., at pre-set intervals, responsive to one or more mood changes during a consultation period) during the consultation period. For example, at the beginning of a consultation period, subject 38 may be enthusiastic. As such, coaching component 32 may determine a course of action to maintain and take advantage of the enthusiasm. In this example, subject 38's mood may change to overwhelmed midway through the consultation period due to an intensity, complexity, or difficulty of the course of action. As such, coaching component 32 may determine a new course of action to alleviate subject 38's discomfort.

In some embodiments, coaching component 32 is configured to determine a phenotype corresponding to subject 38 based on data provided by communications component 26. In some embodiments, the phenotypes include one or more of analyst, fighter, optimist, sensitive, and/or other phenotypes. In some embodiments, coaching component 32 is configured to determine a method of communication, topics of discussion, and/or other information based on the determined phenotype of subject 38. In some embodiments, the determined course of action varies based on consultant 40. For example, a determined course of action for consultant 40 may include a referral to a relevant service (e.g., mental health, hospital, general practitioner, etc.), a coping strategy, one or more therapy prescriptions, one or more educational materials, and/or other information.

For example, the method of communication with an optimist phenotype may include having friendly and informal conversations, building trusting relationships, and not being too serious or dramatic regarding subject 38's condition. In this example, topics of discussion may include stories of how other individuals have dealt with the condition, setting and reaching flexible goals, discussing the benefits of a treatment, and/or other topics.

As another example, the method of communication with an analyst phenotype may include speaking in a factual and structured way, helping subject 38 feel knowledgeable about their condition, acknowledging subject 38's expertise and actively involving them as part of a care team. In this example, topics of discussion may include information related to a care plan (e.g., effects, side effects, alternatives), sharing knowledge and skill to help subject 38 remain stable, using visual aids to show progress, and/or other topics.

In yet another example, the method of communication with a fighter phenotype may include being clear and straightforward, focusing on action rather an understanding, and making subject 38 feel in charge. In this example, topics of discussion may include specific action points, emphasis on expected benefits, review and praise of progress, and/or other topics.

In another example, the method of communication with a sensitive phenotype may include being calm, gentle, emphatic and reassuring, providing enough information (e.g., without providing every detail), and/or other methods. In this example, topics of discussion may include acknowledging subject 38's situation, subject 38's concerns, offering professional guidance on coping with a condition, care plan expectations and side effects, and/or other topics.

In some embodiments, coaching component 32 is configured to determine subject 38's coping style based on data provided by communications component 26. In some embodiments, coaching component 32 is configured to, responsive to an identification of subject 38's coping style, determine a course of action for interacting with subject 38. In some embodiments, responsive to subject 38's coping style being problem focused, coaching component 32 is configured to identify coping strategies for subject 38, identify problems requiring an approach other than problem-solving, identify one or more ways for subject 38 to express their emotions to relieve frustration and identify helpful strategies, and/or take other actions. In some embodiments, responsive to subject 38's coping style being emotion focused, coaching component 32 is configured to (i) identify health problems with a corresponding degree of urgency, (ii) select one or more controllable problems for addressing for a particular time period, (iii) provide one or more problem-solving strategies to be selected by subject 38, and/or take other actions. In some embodiments, responsive to subject 38's coping style being distraction based, coaching component 32 is configured to (i) determine whether subject 38 acknowledges their health problems, (ii) facilitate subject 38 to select one or more health problems to be addressed, (iii) provide one or more problem-solving strategies to be selected by subject 38, and/or take other actions.

In some embodiments, coaching component 32 is configured to (i) determine a preliminary course of action based on semantic analysis of one or more previous interactions with the subject and (ii) automatically adjust, during the consultation period, the preliminary course of action based on the detected mood. In some embodiments, the second time precedes the first time. For example, coaching component 32 is configured to (i) semantically analyze one or more previous coaching session transcripts of subject 38, (ii) determine a preliminary course of action based on one or more topics discussed during the one or more previous coaching sessions, one or more psychosocial needs identified during the one or more previous coaching sessions, and/or other information, (iii) responsive to subject 38 not appearing to respond well to the preliminary course of action, coaching component 32 is configured to automatically adjust, in real-time, the preliminary course of action based on the detected mood, the one or more words or phrases, and/or other information obtained in real-time. In this example, subject 38 may have shown symptoms of depression during a previous coaching session. As such, coaching component 32 may determine adding a daily (e.g., routine) exercise regimen as a preliminary course of action; however, during a subsequent coaching session, it may be determine that disease and related symptoms (e.g., breathlessness and fatigue) pose an impediment to subject 38's physical activities thus causing subject 38 to be de-motivated and further depressed. As such, coaching component 28 may adjust the preliminary course of action to include (i) a prescribed diet (e.g., establish healthy eating habits, add dietary supplements, etc.) and (ii) set an easily attainable exercise goal.

In some embodiments, coaching component 32 is configured to determine the course of action for the consultation period for interacting with subject 38 based on one or more population statistics. In some embodiments, coaching component 32 is configured to determine the preliminary course of action based on treatments generally offered to a population having one or more similar attributes as subject 38. For example, the population may be affected by the same disease, the population may be in the same age group, the population may have undergone similar procedures (e.g., surgery), and/or other population statistics.

In some embodiments, coaching component 32 may be and/or include a prediction model. As an example, the prediction model may include a neural network or other prediction model (described above) that is trained and utilized for determining and/or adjusting a course of action (described above). In some embodiments, coaching component 32 may adjust the course of action based on historical and real-time data corresponding to the mood of subject 38. For example, coaching component may adjust the course of action based on how subject 38's mood has historically changed responsive to an interaction incorporating a similar course of action. As another example, coaching component 32 may predict how subject 38's mood will be affected responsive to an upcoming interaction incorporating a particular course of action. In yet another example, coaching component 32 may update the prediction models based on real-time mood information of subject 38. In this example, subject 38's mood response is continuously recorded and updated based on exposure to interaction incorporating different courses of action.

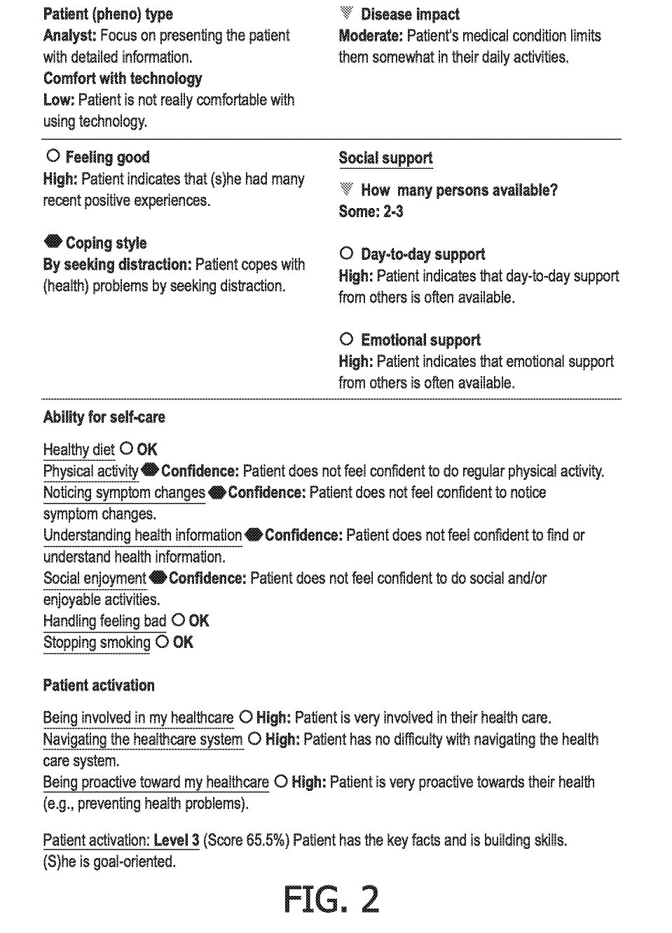

Presentation component 34 is configured to provide, via user interface 20, one or more cues for presentation to consultant 40 during the consultation period. In some embodiments, the cues indicate the determined course of action to be taken by consultant 40 for interacting with subject 38. By way of a non-limiting example, FIG. 2 illustrates a patient coaching summary, in accordance with one or more embodiments. As shown in FIG. 2, presentation component 34 provides visual information regarding subject 38's phenotype, comfort with technology, disease impact, coping style, social support, ability for self-care, patient activation, and/or other information. Presentation component 34 is configured to emphasize one or more psychosocial needs of subject 38 by incorporating one or more different colors and/or shapes. In some embodiments, the emphasis is based on an urgency of the one or more psychosocial needs, a degree of difficulty in handling the one or more psychosocial needs, and/or other factors. For example, responsive to subject 38 indicating low confidence in performing regular physical activity, noticing symptom changes, understanding health information, and social enjoyment, presentation component 34 is configured to emphasize the psychosocial needs by changing an indicator color corresponding to physical activity, noticing symptom changes, understanding health information and social enjoyment to red. In some embodiments, presentation component 34 is configured to effectuate, via user interface 20, presentation of local activities (e.g., to help subject 38), links to relevant websites, videos, or other resources, and/or other information.

In some embodiments, presentation component 34 is configured to generate augmented reality content based on the determined course of action and overlay the augmented reality content on the live view of the real-world environment for presentation to consultant 40 during the consultation period. The augmented reality presentation may, for example, comprise a live view of the real-world environment and one or more augmentations to the live view. The augmentations may comprise content provided by coaching component 32 (e.g., determined course of action), other content related to one or more aspects in the live view, or other augmentations.

As an example, the augmented reality content may comprise visual or audio content (e.g., text, images, audio, video, etc.) generated at a remote computer system based on the determined course of action (e.g., as determined by coaching component 32), and presentation component 34 may obtain the augmented reality content from the remote computer system. In some embodiments, presentation component 34 may overlay, in the augmented reality presentation, the augmented reality content on a live view of the real-world environment. In an embodiment, the presentation of the augmented reality content (or portions thereof) may occur automatically, but may also be “turned off” a the user (e.g., by manually hiding the augmented reality content or portions thereof after it is presented, by setting preferences to prevent the augmented reality content or portions thereof from being automatically presented, etc.). As an example, consultant 40 may choose to reduce the amount of automatically-displayed content via user preferences (e.g., by selecting the type of information consultant 40 desires to be automatically presented, by selecting the threshold amount of information that is to be presented at a given time, etc.). By way of a non-limiting example, consultant 40 may be wearing Google Glass. In this example, consultant 40 may be provided, on the prism display, with one or more of an indicator indicative of a mood change of subject 38 with respect to a topic of discussion, one or more instructions, questions, discussion topics to be asked from subject 38 to positively affect subject 38's mood, and/or other augmented reality content.

In some embodiments, presentation component 34 is configured to output the augmented-reality-enhanced view on user interface 20 (e.g., Google Glass, a display screen) or on any other user interface device. In some embodiments, presentation component 34 outputs the augmented-reality-enhanced view in response to a change in the mood of subject 38.

In some embodiments, presentation component 34 is configured to provide an audio or visual summary of one or more previous interactions of subject 38 to consultant 40 prior to the interaction during the first time.

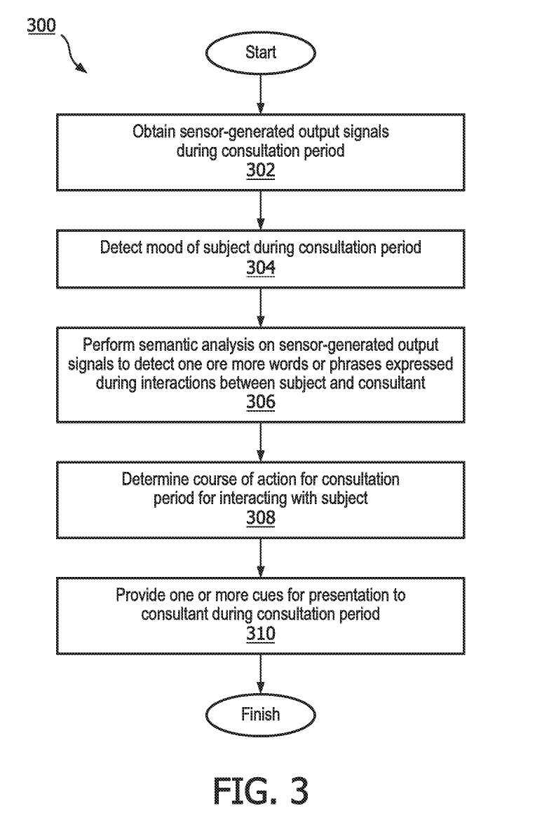

FIG. 3 illustrates a method 300 for facilitating determination of a course of action for an individual. Method 300 may be performed with a system. The system comprises one or more sensors and one or more processors, or other components. The processors are configured by machine readable instructions to execute computer program components. The computer program components include a communications component, a mood determination component, a content analysis component, a coaching component, a presentation component, or other components. The operations of method 300 presented below are intended to be illustrative. In some embodiments, method 300 may be accomplished with one or more additional operations not described, or without one or more of the operations discussed. Additionally, the order in which the operations of method 300 are illustrated in FIG. 3 and described below is not intended to be limiting.

In some embodiments, method 300 may be implemented in one or more processing devices (e.g., a digital processor, an analog processor, a digital circuit designed to process information, an analog circuit designed to process information, a state machine, or other mechanisms for electronically processing information). The devices may include one or more devices executing some or all of the operations of method 300 in response to instructions stored electronically on an electronic storage medium. The processing devices may include one or more devices configured through hardware, firmware, or software to be specifically designed for execution of one or more of the operations of method 300.

At an operation 302, sensor-generated output signals are obtained during a consultation period. In some embodiments, operation 302 is performed by a processor component the same as or similar to communications component 26 (shown in FIG. 1 and described herein).

At an operation 304, a mood of a subject is detected based on the sensor-generated output signals during the consultation period. In some embodiments, operation 304 is performed by a processor component the same as or similar to mood determination component 28 (shown in FIG. 1 and described herein).

At an operation 306, semantic analysis is performed on the sensor-generated output signals to detect one or more words or phrases expressed during interactions between the subject and a consultant. In some embodiments, operation 306 is performed by a processor component the same as or similar to content analysis component 30 (shown in FIG. 1 and described herein).

At an operation 308, a course of action is determined for the consultation period for interacting with the subject. In some embodiments, the determination of the course of action is determined during the consultation period based on the detected mood and the one or more words or phrases. In some embodiments, operation 308 is performed by a processor component the same as or similar to coaching component 32 (shown in FIG. 1 and described herein).

At an operation 310, one or more cues are provided, via a user interface, for presentation to a consultant during the consultation period. In some embodiments, the cues indicate the determined course of action to be taken by the consultant for interacting with the subject. In some embodiments, operation 310 is performed by a processor component the same as or similar to presentation component 34 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A system configured to facilitate determination of a course of action for a subject, the system comprising:
one or more sensors configured to generate, during a consultation period, output signals conveying information related to interactions between the subject and a consultant, the one or more sensors including at least a sound sensor and an image sensor; and
one or more processors configured by machine-readable instructions to:
obtain, from the one or more sensors, the sensor-generated output signals during the consultation period;
detect, based on the sensor-generated output signals, a mood of the subject during the consultation period;
determine a course of action for the subject during the consultation period based on the detected mood; and
provide, via a user interface, one or more cues for presentation to the consultant during the consultation period, the cues indicating the determined course of action to be taken by the consultant for interacting with the subject.

2. The system of claim 1, wherein the one or more processors are configured to detect the mood of the subject based on one or more of a tone of voice of the subject, verbal cues, facial expressions of the subject, seat activities of the subject, a heart rate of the subject, a respiration of the subject, or an electrodermal activity of the subject.

3. The system of claim 1, wherein the one or more sensors further comprise one or more of a heart rate sensor, a respiration sensor, a perspiration sensor, an electrodermal activity sensor, or an activity sensor.

4. The system of claim 6, wherein the one or more processors are further configured to (i) receive, from the one or more sensors, a live view of a real-world environment, (ii) generate augmented reality content based on the determined course of action, and (iii) overlay the augmented reality content on the live view of the real-world environment for presentation to the consultant during the consultation period.

5. The system of claim 1, wherein the one or more processors are further configured to (i) determine a preliminary course of action based on semantic analysis of one or more previous interactions with the subject and (ii) automatically adjust, during the consultation period, the preliminary course of action based on the detected mood.

6. The system of claim 1, wherein the one or more processors are further configured to (i) perform semantic analysis on the sensor-generated output signals to detect one or more words or phrases expressed during the interactions between the subject and the consultant and (ii) determine the course of action based on the one or more words or phrases.

7. A method for facilitating determination of a course of action for subject with a system, the system comprising one or more sensors and one or more processors, the method comprising:
obtaining, from the one or more sensors, output signals conveying information related to interactions between the subject and a consultant during a consultation period, the one or more sensors including at least a sound sensor and an image sensor;
detecting, based on the sensor-generated output signals, a mood of the subject during the consultation period;
determining, with the one or more processors, a course of action for the subject during the consultation period based on the detected mood; and
providing, via a user interface, one or more cues for presentation to the consultant during the consultation period, the cues indicating the determined course of action to be taken by the consultant for interacting with the subject.

8. The method of claim 7, wherein detecting the mood of the subject is based on one or more of a tone of voice of the subject, verbal cues, facial expressions of the subject, seat activities of the subject, a heart rate of the subject, a respiration of the subject, or an electrodermal activity of the subject.

9. The method of claim 7, wherein the one or more sensors further comprise one or more of a heart rate sensor, a respiration sensor, a perspiration sensor, an electrodermal activity sensor, or an activity sensor.

10. The method of claim 7, further comprising (i) receiving, from the one or more sensors, a live view of a real-world environment, (ii) generating, with the one or more processors, augmented reality content based on the determined course of action, and (iii) overlaying, with the one or more processors, the augmented reality content on the live view of the real-world environment for presentation to the consultant during the consultation period.

11. The method of claim 7, further comprising (i) determining a preliminary course of action based on semantic analysis of one or more previous interactions with the subject and (ii) automatically adjusting, during the consultation period, the preliminary course of action based on the detected mood.

12. The method of claim 7, further comprising (i) performing, with the one or more processors, semantic analysis on the sensor-generated output signals to detect one or more words or phrases expressed during the interactions between the subject and the consultant and (ii) determining, with the one or more processors, the course of action based on the one or more words or phrases.

13. A system configured to facilitate determination of a course of action for a subject, the system comprising:
means for generating, during a consultation period, output signals conveying information related to interactions between the subject and a consultant, the means for generating including at least a sound sensor and an image sensor;
means for obtaining the output signals during the consultation period;
means for detecting, based on the output signals, a mood of the subject during the consultation period;
means for determining a course of action for the subject during the consultation period based on the detected mood; and
means for providing one or more cues for presentation to the consultant during the consultation period, the cues indicating the determined course of action to be taken by the consultant for interacting with the subject.

14. The system of claim 13, wherein detecting the mood of the subject is based on one or more of a tone of voice of the subject, verbal cues, facial expressions of the subject, seat activities of the subject, a heart rate of the subject, a respiration of the subject, or an electrodermal activity of the subject.

15. The system of claim 13, wherein the means for generating output signals further comprises one or more of a heart rate sensor, a respiration sensor, a perspiration sensor, an electrodermal activity sensor, or an activity sensor.

16. The system of claim 13, further comprising (i) means for receiving, from the means for generating output signals, a live view of a real-world environment, (ii) means for generating augmented reality content based on the determined course of action, and (iii) means for overlaying the augmented reality content on the live view of the real-world environment for presentation to the consultant during the consultation period.

17. The system of claim 13, further comprising (i) means for determining a preliminary course of action based on semantic analysis of one or more previous interactions with the subject and (ii) means for automatically adjusting, during the consultation period, the preliminary course of action based on the detected mood.

18. The system of claim 13, further comprising (i) means for performing semantic analysis on the sensor-generated output signals to detect one or more words or phrases expressed during the interactions between the subject and the consultant and (ii) means for determining the course of action based on the one or more words or phrases.
